(19) United States
(12) Patent Application Publication
(10) Pub. No.: US 2020/0100832 A1
(43) Pub. Date: Apr. 2, 2020

(54) TREATMENT SYSTEM

(71) Applicant: Olympus Corporation, Tokyo (JP)

(72) Inventors: Mizuki Hanawa, Tokyo (JP); Akihito Kano, Tokyo (JP)

(73) Assignee: Olympus Corporation, Tokyo (JP)

(21) Appl. No.: 16/699,819

(22) Filed: Dec. 2, 2019

Related U.S. Application Data
(63) Continuation of application No. PCT/JP2017/021941, filed on Jun. 14, 2017.

(51) Int. Cl. A61B 18/14

(52) U.S. Cl. CPC A61B 18/14; A61B 2018/00994

(57) ABSTRACT

The disclosed technology is directed to a treatment system comprises a power supply apparatus and a treatment instrument configured to communicate electrically so as to perform an operation on a biological tissue. The treatment instrument includes an end effector that transmits a high-frequency current to the biological tissue. An ultrasonic transducer is disposed in the treatment instrument. The ultrasonic transducer generates ultrasonic vibration when a second electrical energy is supplied. The ultrasonic transducer delivers the ultrasonic vibration to the end effector. The power supply apparatus includes a processor that supplies the first electrical energy to the end effector with a first voltage waveform about which a crest factor is equal to or lower than 1.5 and controls output of the first electrical energy in a manner that the biological tissue is degenerated and coagulated due to flowing of the high-frequency current in the biological tissue with no discharge occurs.

### CROSS-REFERENCE TO RELATED APPLICATION

This application is a continuation application of PCT Application No. PCT/JP 2017/021941 filed on Jun. 14, 2017, which is hereby incorporated by reference in its entirety.

### TECHNICAL FIELD

The disclosed technology relates to a power supply apparatus used with a treatment instrument including an end effector that can give a high-frequency current to biological tissue and an ultrasonic transducer that transmits generated ultrasonic vibration to the end effector.

### DESCRIPTION OF THE RELATED ART

AUS Patent Publication 2013/0123820 A1-discloses a treatment system including a treatment instrument having an end effector and a return electrode that is a separate unit from the treatment instrument. In this treatment system, an ultrasonic transducer is disposed in the treatment instrument. In a typical treatment using this treatment system, the end effector is brought into contact with biological tissue that is a treatment target in the state in which first electrical energy, or high-frequency power, is supplied to the end effector and the return electrode and simultaneously second electrical energy is supplied to the ultrasonic transducer. At this time, a high-frequency current is given to the biological tissue due to the supply of the first electrical energy to the end effector and the return electrode. In addition, ultrasonic vibration is generated in the ultrasonic transducer due to the supply of the second electrical energy to the ultrasonic transducer. Then, at the end effector, the ultrasonic vibration is transmitted to the biological tissue simultaneously with the high-frequency current. As described hereinbefore, the biological tissue is incised simultaneously with coagulation in the state in which the high-frequency current and the ultrasonic vibration are simultaneously given to the biological tissue.

In the typical treatment using a treatment system like that in US Patent Publication 2013/0123820 A1, it is required that incision and hemostasis are properly carried out for a part coagulated and incised by using the high-frequency current and the ultrasonic current in biological tissue.

### BRIEF SUMMARY OF EMBODIMENTS

The disclosed technology has been made in view of the foregoing.

One aspect of the disclosed technology is directed to a treatment system comprises a power supply apparatus and a treatment instrument configured to communicate electrically with the power supply apparatus so as to perform an operation on a biological tissue. The treatment instrument includes an end effector that transmits a high-frequency current delivered by a first electrical energy to the biological tissue. An ultrasonic transducer is disposed in the treatment instrument. The ultrasonic transducer generates ultrasonic vibration when a second electrical energy is supplied. The ultrasonic transducer delivers the ultrasonic vibration to the end effector. The power supply apparatus includes a processor that in a first control mode, the processor supplies the first electrical energy to the end effector with a first voltage waveform about which a crest factor is equal to or lower than 1.5, and controls output of the first electrical energy in such a manner that the biological tissue is degenerated and coagulated by Joule heat generated due to flowing of the high-frequency current in the biological tissue with no discharge occurs when a gap is made between the end effector and the biological tissue. In a second control mode, the processor supplies the first electrical energy to the end effector with a second voltage waveform that generates a discharge between the end effector and the biological tissue, and causes the second electrical energy to be supplied to the ultrasonic transducer in such a manner as to obtain a state in which the biological tissue is incised and/or is degenerated by friction heat attributed to the ultrasonic vibration at the end effector. The processor carries out switching to the second control mode based on satisfaction of a predetermined condition in the first control mode.

Another aspect of the disclosed technology is directed to a treatment system comprises a power supply apparatus, a return electrode configured to be detachably attached to the power supply and a treatment instrument configured to communicate electrically with the power supply apparatus so as to perform an operation on a biological tissue. The treatment instrument includes a housing, a shaft, and a rod member having an end effector all of which being attached to one another to define the treatment instrument. The end effector transmits a high-frequency current delivered by a first electrical energy to the biological tissue. An ultrasonic transducer is disposed in the treatment instrument. The ultrasonic transducer generates ultrasonic vibration delivered by a second electrical energy to the end effector. The power supply apparatus includes a processor that in a first control mode, the processor supplies the first electrical energy to the end effector with a first voltage waveform about which a crest factor is equal to or lower than 1.5, and controls output of the first electrical energy in such a manner that the biological tissue is degenerated and coagulated by Joule heat generated due to flowing of the high-frequency current in the biological tissue with no discharge occurs when a gap is made between the end effector and the biological tissue. In a second control mode, the processor supplies the first electrical energy to the end effector with a second voltage waveform that generates a discharge between the end effector and the biological tissue and causes the second electrical energy to be supplied to the ultrasonic transducer in such a manner as to obtain a state in which the biological tissue is incised and/or is degenerated by friction heat attributed to the ultrasonic vibration at the end effector. The processor carries out switching to the second control mode based on satisfaction of a predetermined condition in the first control mode.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is a schematic diagram depicting a treatment system according to a first embodiment.

FIG. 2 is a block diagram schematically depicting a configuration that supplies electrical energy to a treatment instrument according to the first embodiment.

FIG. 3 is a schematic diagram depicting one example of a first voltage waveform of an output voltage to an end effector and a return electrode according to the first embodiment.

FIG. 4 is a schematic diagram depicting one example of a second voltage waveform of the output voltage to the end effector and the return electrode according to the first embodiment.

FIG. 5 is a flowchart depicting processing executed by a processor in output control of the electrical energy to the treatment instrument according to the first embodiment.

FIG. 6 is a schematic diagram depicting one example of switching between a first control mode and a second control mode over time in processing using the treatment system according to the first embodiment.

### DETAILED DESCRIPTION OF THE EMBODIMENTS

In the following description, various embodiments of the technology will be described. For purposes of explanation, specific configurations and details are set forth in order to provide a thorough understanding of the embodiments. However, it will also be apparent to one skilled in the art that the technology disclosed herein may be practiced without the specific details. Furthermore, well-known features may be omitted or simplified in order not to obscure the embodiment being described.

An object of the disclosed technology is to provide a treatment apparatus with which incision and hemostasis are properly carried out for a part given a high-frequency current and ultrasonic vibration in treatment of coagulating and incising biological tissue by the high-frequency current and the ultrasonic vibration.

### First Embodiment

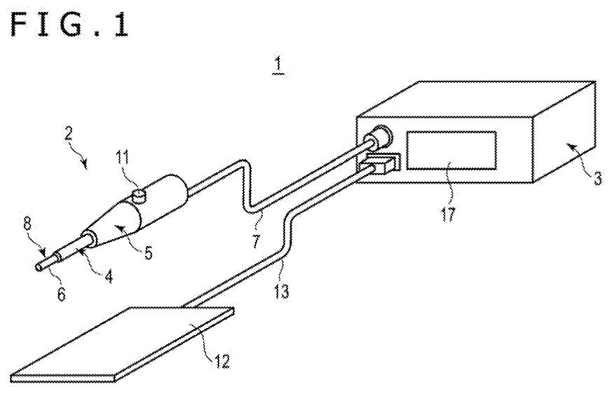

A first embodiment of the disclosed technology will be described with reference to FIG. 1 to FIG. 6.

FIG. 1 is a diagram depicting a treatment system 1 of the present embodiment. As depicted in FIG. 1, the treatment system 1 includes a treatment instrument 2 and a power supply apparatus 3. The treatment instrument 2 includes a tubular shaft 4, a housing 5 that can be held, and an end effector 6. The housing 5 is joined to one side of the shaft 4 regarding a direction along a center axis of the shaft 4. Furthermore, in the present embodiment, a center axis of the housing 5 is coaxial with or substantially coaxial with the center axis of the shaft 4. Here, a side on which the housing 5 is located with respect to the shaft 4 regarding the direction along the center axis of the shaft 4 is defined as a proximal side and the opposite side to the proximal side is defined as a distal side. One end of a cable 7 is connected to a proximal portion of the housing 5. The other end of the cable 7, defined as a second cable, can be detachably connected to the power supply apparatus 3.

Furthermore, in the treatment instrument 2, a rod member 8 passes inside the shaft 4 from the inside of the housing 5 and is extended toward the distal side. The end effector 6 is formed from part of the rod member 8. In the present embodiment, the rod member 8 protrudes toward the distal side from a distal end of the shaft 4 and the end effector 6 is formed by the protrusion part from the shaft 4 in the rod member 8. The rod member 8 is formed from a material with high vibration transmissibility, such as a titanium alloy. Furthermore, the end effector 6 has electrical conductivity. The end effector 6 is formed into any of hook shape, spatula shape, ball shape, and so forth, for example.

An operation button 11 is disposed on the housing 5 as an operation member. With the operation button 11, operation to cause electrical energy to be supplied to the treatment instrument 2 as described hereinafter can be input. In a certain embodiment example, instead of the operation button 11 or in addition to the operation button 11, a foot switch or the like that is a separate unit from the treatment instrument 2 may be disposed as an operation member that can input operation to cause electrical energy to be supplied to the treatment instrument 2. Furthermore, a return electrode 12 that is a separate unit from the treatment instrument 2 is disposed in the treatment system 1. The return electrode 12 is detachably connected to the power supply apparatus 3 through a cable 13 defined as a first cable.

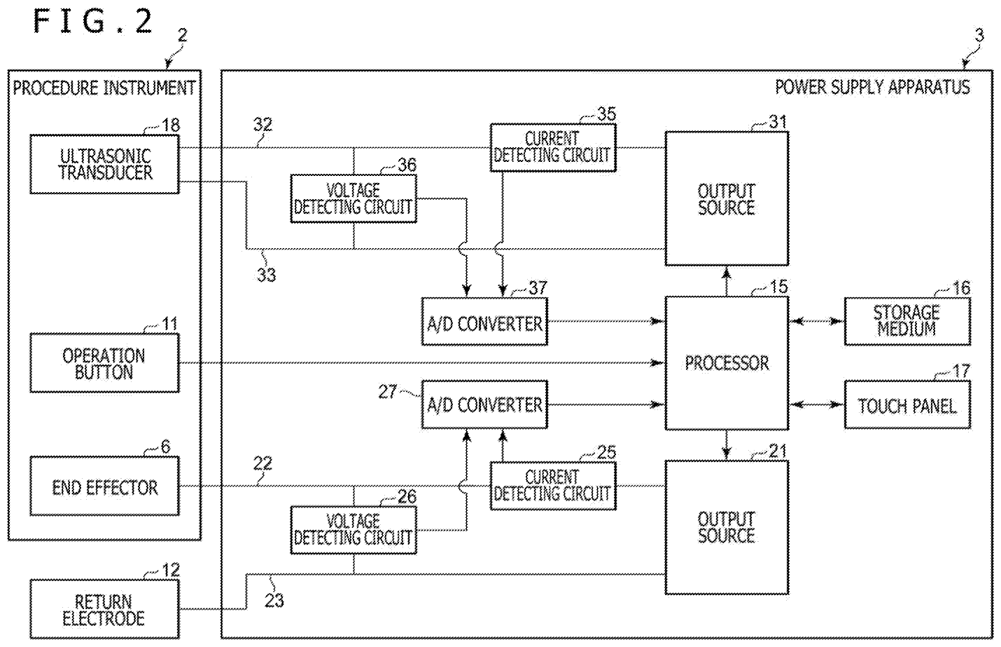

FIG. 2 is a diagram depicting a configuration that supplies electrical energy to the treatment instrument 2. As depicted in FIG. 2, the power supply apparatus 3 includes a processor 15, or controller, and a storage medium 16. The processor 15 is formed from an integrated circuit including a CPU (Central Processing Unit), ASIC (Application Specific Integrated Circuit), FPGA (Field Programmable Gate Array), or the like or circuitry or the like. Only one processor 15 may be disposed in the power supply apparatus 3 or multiple processors 15 may be disposed in the power supply apparatus 3. In the present embodiment, the processor 15 configures at least part of a power supply apparatus that controls the treatment system 1. Processing in the processor 15 is executed in accordance with a program stored in the processor 15 or the storage medium 16. Furthermore, in the storage medium 16, processing programs used in the processor 15, parameters used in arithmetic operation in the processor 15, functions, tables, and so forth are stored.

The processor 15 determines whether or not operation input is being carried out with the operation button 11, or operation member, that is, whether the operation input with the operation button 11 is ON or OFF. In a certain embodiment example, a switch (not depicted) is disposed inside the housing 5 in conformity with the operation button 11 and switching is carried out between ON and OFF in the switch corresponding to operation with the operation button 11.

The power supply apparatus 3 includes an output source 21, high-frequency power supply. The output source 21 includes a waveform generator, a conversion circuit, a relay circuit, a transformer, and so forth and forms a drive circuit, or high-frequency drive circuit. The output source 21 can convert power from a battery power supply, outlet power supply, or the like to high-frequency power, or high-frequency electrical energy, that is first electrical energy and output the first electrical energy. The output source 21 is electrically connected to the end effector 6 through an electrical path 22 and is electrically connected to the return electrode 12 through an electrical path 23. The electrical path 22 is extended through the inside of the cable 7, for example, and the electrical path 23 is extended through the inside of the cable 13 defined as first cable, for example.

The first electrical energy output from the output source 21 is supplied to the end effector 6 and the return electrode 12 through the electrical paths 22 and 23. Due to the supply of the first electrical energy, or high-frequency power, to the end effector 6 and the return electrode 12, the end effector 6 and the return electrode 12 function as electrodes having potentials different from one another. This makes it possible to cause a high-frequency current to flow between the end effector 6 and the return electrode 12 and give the high-frequency current to biological tissue or the like. When operation input is carried out with the operation button 11, the processor 15 controls the output from the output source 21 and controls the supply of the first electrical energy to the end effector 6 and the return electrode 12.

Furthermore, a current detecting circuit 25, a voltage detecting circuit 26, and an A/D (Analog-to-Digital) converter 27 are disposed in the power supply apparatus 3. The current detecting circuit 25 detects an output current I from the output source 21 to the end effector 6 and the return electrode 12, and the voltage detecting circuit 26 detects an output voltage V to the end effector 6 and the return electrode 12. The A/D converter 27 converts an analog signal that indicates the current value of the output current I detected by the current detecting circuit 25 and an analog signal that indicates the voltage value of the output voltage V detected by the voltage detecting circuit 26 to digital signals, and transmits the converted digital signals to the processor 15. Thereby, the processor 15 acquires information relating to the output current I and the output voltage V from the output source 21.

Moreover, based on the output current I and the output voltage V from the output source 21, the processor 15 calculates impedance Z between the end effector 6 and the return electrode 12 as the impedance of the circuit in which the high-frequency current, or output current I, flows. Here, in the state in which the high-frequency current is being given to biological tissue by the end effector 6, the impedance Z between the end effector 6 and the return electrode 12 changes corresponding to the impedance of the biological tissue, and the impedance Z increases when the impedance of the biological tissue increases. For this reason, the impedance Z is a parameter relating to the first electrical energy and is a parameter that changes corresponding to the state of the biological tissue. Furthermore, the processor 15 calculates output power P from the output source 21 based on the output current I and the output voltage V from the output source 21. The processor 15 controls the output of the first electrical energy from the output source 21 to the end effector 6 and the return electrode 12 based on the output current I, the output voltage V, the impedance Z, the output power P, and so forth.

An ultrasonic transducer 18 is disposed in the treatment instrument 2. The ultrasonic transducer 18 is connected to the rod member 8 inside the housing 5. Furthermore, the power supply apparatus 3 includes an output source 31, or ultrasonic power supply. The output source 31 includes a waveform generator, a conversion circuit, a relay circuit, a transformer, and so forth and forms a drive circuit, or ultrasonic drive circuit. The output source 31 can convert power from a battery power supply, outlet power supply, or the like to second electrical energy and output the second electrical energy. The output source 31 is connected to the ultrasonic transducer 18 through electrical paths 32 and 33. Each of the electrical paths 32 and 33 is extended through the inside of the cable 7, for example.

The second electrical energy output from the output source 31 is supplied to the ultrasonic transducer 18 through the electrical paths 32 and 33. At this time, alternating-current power with a certain frequency in a predetermined frequency range is supplied to the ultrasonic transducer 18 as the second electrical energy. When operation input is carried out with the operation button 11, the processor 15 controls the output from the output source 31 and controls the supply of the second electrical energy to the ultrasonic transducer 18.

Due to the supply of the second electrical energy, or alternating-current power, to the ultrasonic transducer 18, the ultrasonic transducer 18 converts the electrical energy to vibration energy by a piezoelectric element (not depicted) or the like to generate ultrasonic vibration. The ultrasonic vibration generated by the ultrasonic transducer 18 is transmitted to the end effector 6 through the rod member 8. Due to the transmission of the ultrasonic vibration to the end effector 6, the end effector 6 vibrates and the end effector 6 becomes capable of giving the transmitted ultrasonic vibration to biological tissue or the like. At this time, the rod member 8 including the end effector 6 vibrates at the certain frequency in the predetermined frequency range. In the present embodiment, a vibration direction of the rod member 8 is parallel to or substantially parallel to the center axis of the housing 5.

Furthermore, a current detecting circuit 35, a voltage detecting circuit 36, and an A/D converter 37 are disposed in the power supply apparatus 3. The current detecting circuit 35 detects an output current I′ from the output source 31 to the ultrasonic transducer 18 and the voltage detecting circuit 36 detects an output voltage V′ to the ultrasonic transducer 18. The A/D converter 37 converts an analog signal that indicates the current value of the output current I′ detected by the current detecting circuit 35 and an analog signal that indicates the voltage value of the output voltage V′ detected by the voltage detecting circuit 36 to digital signals, and transmits the converted digital signals to the processor 15. Thereby, the processor 15 acquires information relating to the output current I′ and the output voltage V′ from the output source 31.

Moreover, based on the output current I′ and the output voltage V′ from the output source 31, the processor 15 calculates impedance Z′ of the ultrasonic transducer 18 as the impedance of the circuit in which the output current I′ flows. The impedance Z′ of the ultrasonic transducer 18 is a parameter relating to the second electrical energy and is a parameter that changes corresponding to the state of biological tissue. Furthermore, the processor 15 calculates output power P′ from the output source 31 based on the output current I′ and the output voltage V′ from the output source 31. The processor 15 controls the output of the second electrical energy from the output source 31 to the ultrasonic transducer 18 based on the output current I′, the output voltage V′, the impedance Z′, the output power P′, and so forth. In a certain embodiment example, the processor 15 carries out PLL control (Phase Locked Loop control) to the state in which the phase difference between the output current I′ and the output voltage V′ is eliminated.

Furthermore, in the present embodiment, a touch panel 17 is disposed in the power supply apparatus 3. The touch panel 17 functions as an input portion that enables input of setting relating to the output from each of the output sources 21 and 31, such as the output level of each of the output sources 21 and 31, for example. In addition, the touch panel 17 functions also as a display portion that displays information relating to the output from each of the output sources 21 and 31, such as the output current I and the output voltage V from the output source 21 and the output current I′ and the output voltage V′ from the output source 31, for example.

Next, the power supply apparatus contains the processor 15 of the present embodiment and operation and effects of the treatment system 1 will be described. When carrying out treatment by using the treatment system 1, an operator sets the return electrode 12 on a subject such as a human body and holds the housing 5. Then, the operator inserts the end effector 6 into a body cavity such as an abdominal cavity and disposes the end effector 6 near biological tissue that is a treatment target. Then, the operator carries out operation input at the operation button 11 in the state in which the end effector 6 is located near the biological tissue. Due to turning of the operation input at the operation button 11 to ON, the processor 15 causes electrical energy to be output from the power supply apparatus 3 as described hereinafter. Then, in the state in which the electrical energy is being output from the power supply apparatus 3, the end effector 6 is brought close to or into contact with the biological tissue that is the treatment target, and the treatment of the biological tissue is carried out as described hereinafter by using high-frequency current and ultrasonic vibration that are treatment energy.

The processor 15 is capable of controlling the output of the first electrical energy and the output of the second electrical energy in a first control mode and is capable of controlling the output of the first electrical energy and the output of the second electrical energy in a second control mode different from the first control mode. In the first control mode, the processor 15 causes the first electrical energy to be output to the end effector 6 and the return electrode 12 with a first voltage waveform. Furthermore, in the first control mode, the processor 15 does not cause the second electrical energy to be output to the ultrasonic transducer 18. For this reason, in the state in which the output control is being carried out in the first control mode, even when the end effector 6 is brought close to or into contact with biological tissue, only the high-frequency current is given to the biological tissue, and the ultrasonic vibration is not given.

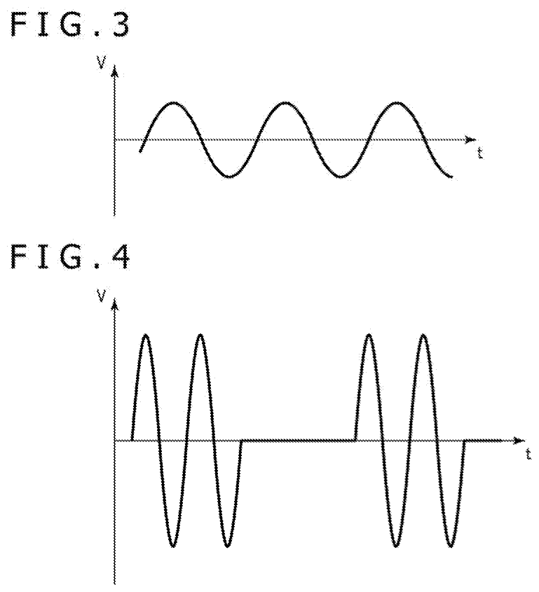

FIG. 3 depicts one example of the first voltage waveform of the output voltage V to the end effector 6 and the return electrode 12. In FIG. 3, a time t is represented on the abscissa axis and the output voltage V is represented on the ordinate axis. In one example of FIG. 3, in the first control mode, the first electrical energy is output continuously over time and the first voltage waveform becomes a continuous wave. Furthermore, with the first voltage waveform, a maximum value, or crest value, of the output voltage V is equal to or smaller than 200 V and the crest factor (CF) of the output voltage V is equal to or lower than 1.5. Here, the crest factor is the value obtained by dividing the maximum value of the output voltage V by an effective value, or RMS (Root Mean Square) value, of the output voltage V. It is understood that, when the first electrical energy is output to the end effector 6 and the return electrode 12 with the first voltage waveform about which the maximum value, or crest value, is equal to or smaller than 200 V and the crest factor is equal to or lower than 1.5, a discharge does not occur between the end effector 6 and biological tissue even when a gap is made between the end effector 6 and the biological tissue.

By bringing the end effector 6 close to or into contact with biological tissue in the state in which the output control is being carried out in the first control mode, the high-frequency current flows between the end effector 6 and the return electrode 12 through the biological tissue. Then, due to the flow of the high-frequency current in the biological tissue, Joule heat is generated due to resistance of the biological tissue and the biological tissue is degenerated and coagulated by the Joule heat.

Here, in the first control mode, the first electrical energy is output with the first voltage waveform. Therefore, even when a gap is made between the end effector 6 and biological tissue, a discharge does not occur between the end effector 6 and the biological tissue as described hereinbefore. For this reason, the high-frequency current sufficient to degenerate and coagulate the biological tissue flows in not only the surface portion but also the deep portion in the biological tissue, and a sufficient quantity of Joule heat is generated to the deep portion in the biological tissue. Thus, when the end effector 6 is brought close to or into contact with the biological tissue in the state in which the output control is being carried out in the first control mode, not only the surface portion but also the deep portion is degenerated and coagulated by the Joule heat in the biological tissue. Because the deep portion is coagulated in addition to the surface portion in the biological tissue as described hereinbefore, the hemostatic performance by the high-frequency current is high in the state in which the output control is being carried out in the first control mode.

Furthermore, in the second control mode, the processor 15 causes the first electrical energy to be output to the end effector 6 and the return electrode 12 with a second voltage waveform different from the first voltage waveform. Moreover, in the second control mode, the processor 15 causes the second electrical energy to be output to the ultrasonic transducer 18. For this reason, in the state in which the output control is being carried out in the second control mode, when the end effector 6 is brought close to or into contact with biological tissue, both high-frequency current and ultrasonic vibration are given to the biological tissue.

FIG. 4 depicts one example of the second voltage waveform of the output voltage V to the end effector 6 and the return electrode 12. In FIG. 4, the time t is represented on the abscissa axis and the output voltage V is represented on the ordinate axis. In one example of FIG. 4, in the second control mode, the first electrical energy is output in an on-and-off manner over time and the first electrical energy is intermittently output. For this reason, the second voltage waveform becomes a burst wave. Furthermore, with the second voltage waveform, the crest factor (CF) of the output voltage V is equal to or higher than 5. Here, if intermittent output of the first electrical energy is carried out, the effective value of the output voltage V over the period in which the output is carried out and the period in which the output is not carried out is used in the calculation of the crest factor of the second voltage waveform. It is understood that, when the first electrical energy is output to the end effector 6 and the return electrode 12 with the second voltage waveform about which the crest factor is equal to or higher than 5, a discharge occurs between the end effector 6 and biological tissue when a gap is made between the end effector 6 and the biological tissue.

The second voltage waveform does not need to be the burst wave depicted in the diagram and it suffices that the crest factor is equal to or higher than 5. Furthermore, it is understood that a discharge occurs in some cases and a discharge does not occur in other cases in a gap between the end effector 6 and biological tissue when the first electrical energy is output to the end effector 6 and the return electrode 12 with a voltage waveform about which the crest factor is higher than 1.5 and is lower than 5. Moreover, it is understood that a discharge occurs in some cases and a discharge does not occur in other cases in a gap between the end effector 6 and biological tissue also when the first electrical energy is output to the end effector 6 and the return electrode 12 with a voltage waveform about which the maximum value, or crest value, is larger than 200 V and the crest factor is equal to or lower than 1.5.

Furthermore, in the second control mode, the processor 15 controls the output of the second electrical energy by constant current control to cause the current value of the output current I′ to become constant or substantially constant over time, for example. Here, amplitude and vibration speed at the end effector 6 based on the ultrasonic vibration change corresponding to the magnitude of the output current I′. For this reason, if the constant current control is carried out regarding the output of the second electrical energy, the amplitude and the vibration speed at the end effector 6 are kept constant or substantially constant over time.

Moreover, in the second control mode, the magnitude of the output current I′ is adjusted to the state in which biological tissue can be incised and/or be degenerated, or coagulated, by friction heat attributed to the ultrasonic vibration at the end effector 6, so that the amplitude and the vibration speed at the end effector 6 are adjusted. In other words, in the second control mode, the output of the second electrical energy to the ultrasonic transducer 18 is controlled to the state in which biological tissue can be incised and/or be degenerated by friction heat attributed to the ultrasonic vibration at the end effector 6.

In the second control mode, the end effector 6 vibrates at high speed due to the ultrasonic vibration. Thus, by bringing the end effector 6 close to or into contact with biological tissue in the state in which the output control is being carried out in the second control mode, the end effector 6 repeats contact with the biological tissue and separation from the biological tissue at high speed. Furthermore, in the second control mode, the first electrical energy is output with the second voltage waveform. Therefore, when a gap is made between the end effector 6 and the biological tissue, a discharge occurs between the end effector 6 and the biological tissue as described hereinbefore. For this reason, due to the repetition of contact with the biological tissue and separation from the biological tissue at high speed by the end effector 6, a discharge occurs in the gap between the end effector 6 and the biological tissue, and a discharged high-frequency current is given to the biological tissue. Due to the giving of the discharged high-frequency current to the biological tissue, the surface portion is incised simultaneously with coagulation in the biological tissue by heat generated due to the discharge.

Also in the state in which the biological tissue is being incised simultaneously with coagulation due to the discharge, the high-frequency current flows in the biological tissue and Joule heat is generated in the biological tissue. Furthermore, the biological tissue is degenerated and coagulated also by the Joule heat. However, because the discharge occurs between the end effector 6 and the biological tissue in the state in which the output control is being carried out in the second control mode, the high-frequency current that flows in the biological tissue is smaller and the Joule heat generated in the biological tissue is smaller compared with the state in which the output control is being carried out in the first control mode.

Moreover, by bringing the end effector 6 close to or into contact with biological tissue in the state in which the output control is being carried out in the second control mode, friction heat is generated between the end effector 6 that vibrates at high speed and the biological tissue. In addition, the biological tissue is incised and/or is degenerated, or coagulated, also by the friction heat attributed to the ultrasonic vibration. Furthermore, in the second control mode, the end effector 6 vibrates at high speed and therefore sticking of the biological tissue to the end effector 6 is effectively prevented.

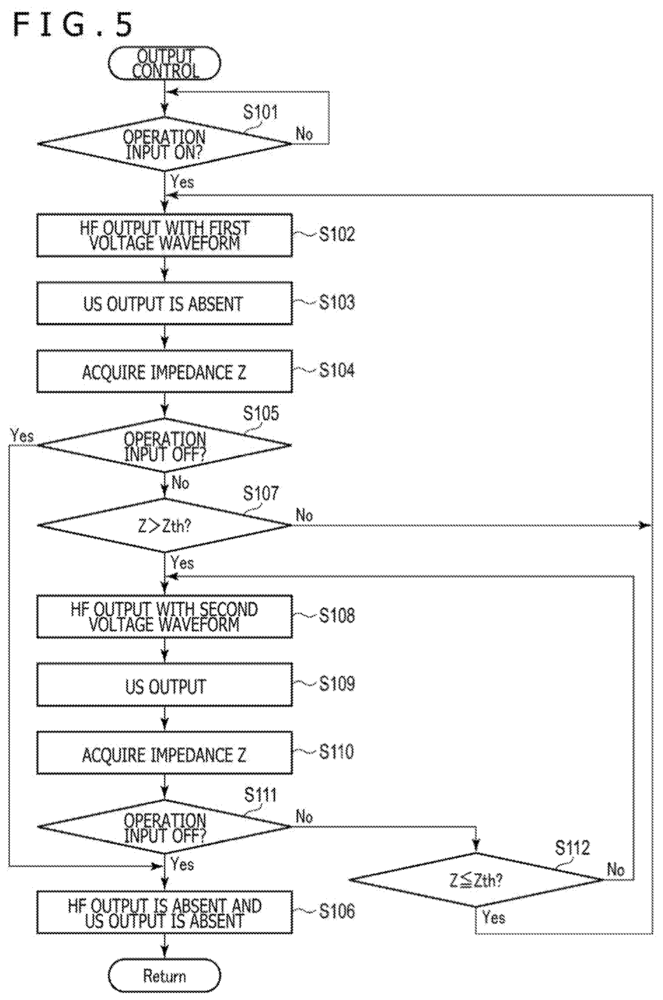

FIG. 5 is a flowchart depicting processing executed by the processor 15 in output control of electrical energy to the treatment instrument 2. As depicted in FIG. 5, the processor 15 determines whether or not operation has been input with an operation member such as the operation button 11, that is, whether operation input with the operation member is ON or OFF in S101. If operation has not been input, No in S101, the processing returns to S101. In other words, the processor 15 waits until operation to cause electrical energy to be supplied to the treatment instrument 2 is input. When operation is input with the operation member, Yes in S101, the processor 15 controls the output of the first electrical energy and the output of the second electrical energy in the first control mode described hereinbefore.

When the output control in the first control mode is started, the processor 15 causes the first electrical energy to be output to the end effector 6 and the return electrode 12 with the first voltage waveform in S102. In other words, HF (high-frequency) output with the first voltage waveform is carried out. Furthermore, in the first control mode, the processor 15 keeps the state in which the output of the second electrical energy to the ultrasonic transducer 18, that is, US (ultrasonic) output, is not carried out in S103. Then, the processor 15 acquires the impedance Z between the end effector 6 and the return electrode 12, that is, the impedance of the circuit in which the output current I flows, as a parameter that changes corresponding to the state of biological tissue in S104.

Then, the processor 15 determines whether or not the operation input with the operation button 11 or the like has been stopped, that is, whether or not the operation input with the operation button 11 has been switched from ON to OFF in S105. If the operation input has been stopped, Yes in S105, the processor 15 stops the output of the first electrical energy, or HF output, to make the state in which the output of the first electrical energy is not carried out and the output of the second electrical energy, or US output, is not carried out in S106. On the other hand, if the operation input with the operation button 11 is continued, No in S105, the processor 15 determines whether or not the impedance Z acquired in S104 is higher than a predetermined threshold Zth in S107. Thereby, whether or not a predetermined condition is satisfied is determined by the processor 15. The predetermined threshold Zth may be set by the touch panel 17 or may be stored in the storage medium 16. Furthermore, the processor 15 sets the predetermined threshold Zth in a range from 500 to 1000 S2 inclusive.

If the impedance Z is equal to or lower than the predetermined threshold Zth, No in S107, the processing returns to S102 and the processor 15 sequentially executes the processing of S102 and the subsequent processing. Therefore, the output control in the first control mode is continued while ON of the operation input is continued and the impedance Z is equal to or lower than the predetermined threshold Zth. If the impedance Z is higher than the predetermined threshold, Yes in S107, the processor 15 determines that the predetermined condition is satisfied, and starts output control in the second control mode. In other words, switching from the first control mode to the second control mode is carried out regarding the output control of the electrical energy to the treatment instrument 2.

When the output control in the second control mode is started, the processor 15 causes the first electrical energy to be output to the end effector 6 and the return electrode 12 with the second voltage waveform in S108. In other words, HF (high-frequency) output with the second voltage waveform is carried out. Furthermore, in the second control mode, the processor 15 causes the second electrical energy to the ultrasonic transducer 18 to be output in S109. At this time, the magnitude of the output current I′ is adjusted to the state in which the biological tissue can be incised and/or be degenerated by friction heat attributed to ultrasonic vibration. In other words, the output control is carried out regarding the US output to the state in which the biological tissue can be incised and/or be degenerated by the friction heat attributed to the ultrasonic vibration. Moreover, also in the second control mode, the processor 15 acquires the impedance Z in S110.

Then, the processor 15 determines whether or not the operation input with the operation button 11 or the like has been stopped in S111. If the operation input has been stopped, Yes in S111, the processor 15 stops the output of the first electrical energy, or HF output, and the output of the second electrical energy, or US output. In other words, the processing proceeds to S106 and the processor 15 makes the state in which the output of the first electrical energy is not carried out and the output of the second electrical energy is not carried out in S106. On the other hand, if the operation input with the operation button 11 is continued, No in S111, the processor 15 determines whether or not the impedance Z acquired in S110 is equal to or lower than the predetermined threshold Zth in S112. The predetermined threshold Zth used in S112 is the same as the predetermined threshold Zth used in the determination of S107.

If the impedance Z is higher than the predetermined threshold Zth, No in S112, the processing returns to S108 and the processor 15 sequentially executes the processing of S108 and the subsequent processing. Therefore, the output control in the second control mode is continued while ON of the operation input is continued and the impedance Z is higher than the predetermined threshold Zth. If the impedance Z is equal to or lower than the predetermined threshold, Yes in S112, the processing proceeds to S102 and the processor 15 sequentially executes the processing of S102 and the subsequent processing. Therefore, if ON of the operation input is continued and the impedance Z has become equal to or lower than the predetermined threshold Zth, the processor 15 carries out switching from the second control mode to the first control mode regarding the output control of the electrical energy to the treatment instrument 2, and carries out the output control in the first control mode again. In this case, the processor 15 carries out the output control in the first control mode again after the output control in the second control mode.

Furthermore, after the switching to the output control in the first control mode is carried out again, if the impedance Z becomes higher than the predetermined threshold Zth, Yes in S107, the processor 15 carries out switching to the output control in the second control mode again. In this case, the processor 15 repeats the output control in the first control mode and the output control in the second control mode alternately. As treatment using the treatment system 1 of the present embodiment, a parenchymal organ, such as a liver, in which a large number of blood vessels are extended internally, or in the deep portion, is coagulated and incised as a treatment target in some cases. In this case, in the state in which output control is being carried out in the first control mode or the second control mode, the end effector 6 is brought close to or into contact with the parenchymal organ, which is biological tissue, and treatment energy is given to the parenchymal organ as described hereinbefore.

Until a certain amount of time elapses from the start of the giving of the treatment energy to a certain part of the parenchymal organ, the temperature is low and the amount of water is also large at the part given the treatment energy and in the vicinity thereof. For this reason, until a certain amount of time elapses from the start of the giving of the treatment energy to the certain part of the parenchymal organ, the impedance Z is low and the processor 15 determines that the impedance Z is equal to or lower than the predetermined threshold Zth. Therefore, the processor 15 continues the output control in the first control mode until a certain amount of time elapses from the start of the giving of the treatment energy to the certain part of the parenchymal organ. Due to this, at the part given the treatment energy in the parenchymal organ, a sufficient high-frequency current flows to the deep portion and not only the surface portion but also the deep portion is degenerated and coagulated by Joule heat until a certain amount of time elapses from the start of the giving of the treatment energy.

Then, when the part given the treatment energy in the parenchymal organ is degenerated to a certain extent by the Joule heat, the temperature rises and dehydration occurs at the part given the treatment energy and in the vicinity thereof. For this reason, the impedance Z rises when a certain amount of time elapses from the start of the giving of the treatment energy to the certain part in the parenchymal organ. Therefore, when a certain amount of time elapses from the start of the giving of the treatment energy to the certain part in the parenchymal organ and the processor 15 determines that the impedance Z is higher than the predetermined threshold Zth, the processor 15 carries out switching to the output control in the second control mode. Furthermore, when the switching to the output control in the second control mode is carried out, at the part given the treatment energy in the parenchymal organ, the surface portion is incised simultaneously with coagulation by heat generated due to a discharge. At this time, at the part given the treatment energy in the parenchymal organ, the surface portion is incised and/or is degenerated, or coagulated, also by friction heat attributed to ultrasonic vibration.

Moreover, when the surface portion is incised and coagulated at the part given the treatment energy in the parenchymal organ, the operator moves the end effector 6 along the surface of the parenchymal organ. Then, the operator brings the end effector 6 close to or into contact with another certain part for which incision and coagulation have not been carried out in the parenchymal organ. At the another certain part to which the end effector 6 has been moved in the parenchymal organ and in the vicinity thereof, the temperature is low and the amount of water is large. For this reason, when the end effector 6 is moved to the another certain part in the parenchymal organ and treatment energy starts to be given to the another certain part, the impedance Z lowers and the processor 15 determines that the impedance Z is equal to or lower than the predetermined threshold Zth. Therefore, when the treatment energy starts to be given to the another certain part in the parenchymal organ, the processor 15 carries out switching to the output control in the first control mode.

Furthermore, treatment is carried out in the manner described hereinbefore also at the another certain part to which the end effector 6 has been moved in the parenchymal organ. Specifically, also at the another certain part to which the end effector 6 has been moved in the parenchymal organ, the surface portion and the deep portion are degenerated and coagulated by Joule heat attributed to the high-frequency current. Then, when the surface portion and the deep portion are degenerated to a certain extent by the Joule heat, switching to the output control in the second control mode is carried out and the surface portion is incised simultaneously with coagulation by heat and ultrasonic vibration attributed to a discharge.

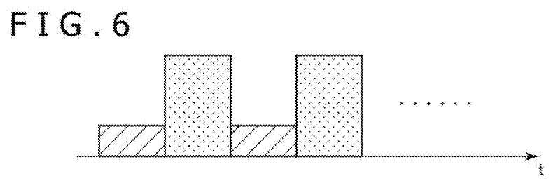

FIG. 6 depicts one example of switching between the first control mode and the second control mode over time in processing using the treatment system 1. As described hereinbefore, because treatment is carried out in a parenchymal organ, the processor 15 carries out output control in the second control mode after output control in the first control mode in the treatment as depicted in FIG. 6. Then, the processor 15 carries out the output control in the first control mode again after the output control in the second control mode. In other words, due to the execution of the treatment in the manner described hereinbefore, the processor 15 repeats the output control in the first control mode and the output control in the second control mode alternately. In FIG. 6, the time t is represented on the abscissa axis. Furthermore, in FIG. 6, periods in which the output control is carried out in the first control mode are depicted by oblique hatching and periods in which the output control is carried out in the second control mode are depicted by dot hatching.

As described hereinbefore, due to use of the treatment system 1 of the present embodiment, at a part given treatment energy in biological tissue, first, the surface portion and the deep portion are degenerated and coagulated by Joule heat attributed to a high-frequency current. Then, after the surface portion and the deep portion are sufficiently coagulated, the surface portion is incised simultaneously with coagulation by heat and ultrasonic vibration attributed to a discharge. Because the incision by the discharge is carried out after coagulation is achieved to the deep portion by the Joule heat, hemostasis is properly carried out for the coagulated and incised part even in treatment of coagulating and incising a parenchymal organ, such as a liver, in which a large number of blood vessels are extended at the deep portion. Furthermore, because the surface portion of the biological tissue is incised simultaneously with coagulation by giving the discharged high-frequency current to the biological tissue, the coagulated and incised part is properly incised. Moreover, in the state in which the surface portion of the biological tissue is being incised simultaneously with coagulation by the discharge, the end effector 6 vibrates due to the ultrasonic vibration. Thus, for example, in the state in which the surface portion of the biological tissue is being incised simultaneously with coagulation by the discharge while the end effector 6 is moved, sticking of the biological tissue to the end effector 6 is prevented.

In the present embodiment, the output of the first electrical energy and the output of the second electrical energy are controlled as described hereinbefore. Therefore, in treatment of coagulating and incising biological tissue by a high-frequency current and ultrasonic vibration, incision and hemostasis are properly carried out for a part given the high-frequency current and the ultrasonic vibration. Furthermore, in the present embodiment, automatic switching is carried out between the first control mode and the second control mode. For this reason, without extracting and removing the end effector 6 to the outside of the body, incising the treatment target while achieving hemostasis by heat to the deep portion of the treatment target by the end effector 6 is carried out by one action.

### MODIFICATION EXAMPLES

In a certain modification example, a temperature sensor (not depicted) is attached to the end effector 6 and a temperature T of the end effector is detected by the temperature sensor. Then, switching is carried out between the first control mode and the second control mode based on the temperature T instead of the impedance Z. In the present modification example, instead of the processing of each of S104 and S110, the processor 15 acquires the temperature T of the end effector 6 from the detection result of the temperature sensor and so forth. Then, instead of the processing of S107, the processor 15 determines whether or not the temperature T is higher than a predetermined threshold Tth. At this time, if the temperature T is equal to or lower than the predetermined threshold Tth, the processing returns to S102 and the processor 15 continues the output control of electrical energy in the first control mode. On the other hand, if the temperature T is higher than the predetermined threshold Tth, the processor 15 determines that the predetermined condition is satisfied. Then, the processing proceeds to S108 and the processor 15 carries out switching to output control in the second control mode.

Furthermore, in the present modification example, instead of the processing of S112, the processor 15 determines whether or not the temperature T is equal to or lower than the predetermined threshold Tth. At this time, if the temperature T is higher than the predetermined threshold Tth, the processing returns to S108 and the processor 15 continues the output control of the electrical energy in the second control mode. On the other hand, if the temperature T is equal to or lower than the predetermined threshold Tth, the processing proceeds to S102 and the processor 15 carries out switching to the output control in the first control mode. The predetermined threshold Tth may be set by the touch panel 17 or may be stored in the storage medium 16. Furthermore, the temperature T is a parameter that changes corresponding to the state of biological tissue similarly to the impedance Z.

As described hereinbefore, until a certain amount of time elapses from the start of the giving of the treatment energy, the temperature is low at the part given the treatment energy and in the vicinity thereof. For this reason, until a certain amount of time elapses from the start of the giving of the treatment energy, the temperature T of the end effector 6 is low and the processor 15 determines that the temperature T is equal to or lower than the predetermined threshold Tth. Therefore, similarly to the embodiment described hereinbefore and so forth, also in the present modification example, the output control in the first control mode is continued and the surface portion and the deep portion of the biological tissue, or parenchymal organ, are degenerated and coagulated by Joule heat attributed to the high-frequency current until a certain amount of time elapses from the start of the giving of the treatment energy.

Furthermore, as described hereinbefore, when the biological tissue is degenerated to a certain extent by the Joule heat, the temperature rises at the part given the treatment energy and in the vicinity thereof. For this reason, when a certain amount of time elapses from the start of the giving of the treatment energy, the temperature T of the end effector 6 rises and the processor 15 determines that the temperature T is higher than the predetermined threshold Tth. Therefore, when the part given the treatment energy is degenerated to a certain extent by the Joule heat, the processor 15 carries out switching to the output control in the second control mode. Then, the surface portion of the biological tissue, or parenchymal organ, is incised simultaneously with coagulation by heat attributed to a discharge and friction heat attributed to the ultrasonic vibration.

Moreover, after the surface portion of the biological tissue is coagulated and incised by the discharge and the ultrasonic vibration, the end effector 6 is moved and the treatment energy starts to be given to another certain part. At this time, the temperature is low at the part to which the end effector 6 has been moved and in the vicinity thereof. For this reason, when the end effector 6 is moved to the another certain part in the biological tissue, the temperature T of the end effector 6 lowers and the processor 15 determines that the temperature T is equal to or lower than the predetermined threshold Tth. Therefore, when the treatment energy starts to be given to the another certain part in the biological tissue, the processor 15 carries out switching to the output control in the first control mode.

Also when switching is carried out between the first control mode and the second control mode based on the temperature T as described hereinbefore, the switching is properly carried out between the first control mode and the second control mode in the treatment of coagulating and incising the biological tissue such as a parenchymal organ similarly to the embodiment described hereinbefore and so forth. Therefore, the present modification example also has the same operation and effects as the embodiment described hereinbefore and so forth.

Furthermore, in still another certain modification example, instead of the processing of each of S104 and S110, the processor 15 acquires the impedance Z′ of the ultrasonic transducer 18 as the impedance of the circuit in which the output current I′ flows. In this case, instead of the processing of S103, the processor 15 causes the ultrasonic transducer 18 to output the second electrical energy with low output power, or minute output power. At this time, the second electrical energy is output with such low output power that biological tissue is not degenerated and incised by the ultrasonic vibration even when the end effector 6 is brought close to or into contact with the biological tissue. In other words, in the present modification example, the US output is carried out in the first control mode. However, it suffices that the impedance Z′ is detected based on the output of the second electrical energy, and the output current I′ from the output source 31 is minute. For this reason, the amplitude and the vibration speed at the end effector 6 are low and the biological tissue is not degenerated due to the ultrasonic vibration and is not incised.

Moreover, in the present modification example, instead of the processing of S107, the processor 15 determines whether or not the impedance Z′ is higher than the predetermined threshold Z′th. At this time, if the impedance Z′ is equal to or lower than the predetermined threshold Z′th, the processing returns to S102 and the processor 15 continues the output control of electrical energy in the first control mode. On the other hand, if the impedance Z′ is higher than the predetermined threshold Z′th, the processor 15 determines that the predetermined condition is satisfied. Then, the processing proceeds to S108 and the processor 15 carries out switching to output control in the second control mode.

Furthermore, in the present modification example, instead of the processing of S111, the processor 15 determines whether or not the impedance Z′ is equal to or lower than the predetermined threshold Z′th. At this time, if the impedance Z′ is higher than the predetermined threshold Z′th, the processing returns to S108 and the processor 15 continues the output control of the electrical energy in the second control mode. On the other hand, if the impedance Z′ is equal to or lower than the predetermined threshold Z′th, the processing proceeds to S102 and the processor 15 carries out switching to the output control in the first control mode. The predetermined threshold Z′th may be set by the touch panel 17 or may be stored in the storage medium 16. Furthermore, the impedance Z′ is a parameter that changes corresponding to the state of biological tissue similarly to the impedance Z.

Here, until the biological tissue is degenerated to a certain extent by Joule heat, the biological tissue is soft and therefore the end effector 6 readily vibrates due to the ultrasonic vibration. For this reason, until the biological tissue is degenerated to a certain extent by the Joule heat, the impedance Z′ is low and it is determined that the impedance Z′ is equal to or lower than the predetermined threshold Z′th. On the other hand, when the biological tissue is degenerated to a certain extent by the Joule heat, the biological tissue is cured and therefore it becomes difficult for the end effector 6 to vibrate based on the ultrasonic vibration. For this reason, when the biological tissue is degenerated to a certain extent by the Joule heat, the impedance Z′ rises and it is determined that the impedance Z′ is higher than the predetermined threshold Z′th. Therefore, also when switching is carried out between the first control mode and the second control mode based on the impedance Z′, the switching is properly carried out between the first control mode and the second control mode in the treatment of coagulating and incising the biological tissue such as a parenchymal organ similarly to the embodiment described hereinbefore and so forth. Thus, the present modification example also has the same operation and effects as the embodiment described hereinbefore and so forth.

Moreover, in yet another certain modification example, the processor 15 acquires a continuation time Y of output control in the first control mode instead of the processing of S104, and the processor 15 acquires a continuation time Y′ of output control in the second control mode instead of the processing of S110. Then, instead of the processing of S107, the processor 15 determines whether or not the continuation time Y is longer than a predetermined threshold Yth. At this time, if the continuation time Y is equal to or lower than the predetermined threshold Yth, the processing returns to S102 and the processor 15 continues the output control of electrical energy in the first control mode. On the other hand, if the continuation time Y is longer than the predetermined threshold Yth, the processor 15 determines that the predetermined condition is satisfied. Then, the processing proceeds to S108 and the processor 15 carries out switching to output control in the second control mode.

Furthermore, in the present modification example, instead of the processing of S112, the processor 15 determines whether or not the continuation time Y′ is longer than the predetermined threshold Y′th. At this time, if the continuation time Y′ is equal to or shorter than the predetermined threshold Y′th, the processing returns to S108 and the processor 15 continues the output control of the electrical energy in the second control mode. On the other hand, if the continuation time Y′ is longer than the predetermined threshold Y′th, the processing proceeds to S102 and the processor 15 carries out switching to the output control in the first control mode. Each of the predetermined thresholds Yth and Y′th may be set by the touch panel 17 or may be stored in the storage medium 16. Furthermore, in a certain embodiment example, each of the predetermined thresholds Yth and Y′th is set to 0.5 seconds to two seconds, for example.

Also in the present modification example, through proper setting of the predetermined thresholds Yth and Y′th, switching is properly carried out between the first control mode and the second control mode in the treatment of coagulating and incising the biological tissue such as a parenchymal organ similarly to the embodiment described hereinbefore and so forth. Thus, the present modification example also has the same operation and effects as the embodiment described hereinbefore and so forth.

Furthermore, only a power supply apparatus 3 is disposed in the embodiment described hereinbefore and so forth. However, in a certain modification example, a power supply apparatus that outputs the first electrical energy and a power supply apparatus that outputs the second electrical energy are separate units. In this case, in the power supply apparatus that outputs the first electrical energy, the output source 21, the current detecting circuit 25, the voltage detecting circuit 26, and the A/D converter 27 described hereinbefore are disposed. In addition, in the power supply apparatus that outputs the second electrical energy, the output source 31, the current detecting circuit 35, the voltage detecting circuit 36, and the A/D converter 37 described hereinbefore are disposed. Moreover, a storage medium and one or more processors are disposed in each of the power supply apparatuses. In addition, the power supply apparatus controls the treatment apparatus 2 by the one or more processors disposed in therein and the processing described hereinbefore is executed.

Moreover, in further certain modification example, one or more processors that execute the processing described hereinbefore are disposed in the treatment instrument 2 and a power supply apparatus that controls the treatment apparatus 2.

In the embodiment described hereinbefore and so forth, in the first control mode, the processor 15 causes the first electrical energy to be output to the end effector 6 with the first voltage waveform about which the crest factor is equal to or lower than 1.5. In the second control mode, the processor 15 causes the first electrical energy to be output to the end effector 6 with the second voltage waveform that generates a discharge between the end effector 6 and biological tissue, and causes the second electrical energy to be output to the ultrasonic transducer 18 to the state in which the biological tissue can be incised and/or be degenerated by friction heat attributed to the ultrasonic vibration described hereinbefore at the end effector 6. The processor 15 carries out switching to the second control mode based on satisfaction of the predetermined condition in the first control mode.

In sum, one aspect of the disclosed technology is directed to a treatment system comprises a power supply apparatus and a treatment instrument configured to communicate electrically with the power supply apparatus so as to perform an operation on a biological tissue. The treatment instrument includes an end effector that transmits a high-frequency current delivered by a first electrical energy to the biological tissue. An ultrasonic transducer is disposed in the treatment instrument. The ultrasonic transducer generates ultrasonic vibration when a second electrical energy is supplied. The ultrasonic transducer delivers the ultrasonic vibration to the end effector. The power supply apparatus includes a processor that in a first control mode, the processor supplies the first electrical energy to the end effector with a first voltage waveform about which a crest factor is equal to or lower than 1.5, and controls output of the first electrical energy in such a manner that the biological tissue is degenerated and coagulated by Joule heat generated due to flowing of the high-frequency current in the biological tissue with no discharge occurs when a gap is made between the end effector and the biological tissue. In a second control mode, the processor supplies the first electrical energy to the end effector with a second voltage waveform that generates a discharge between the end effector and the biological tissue, and causes the second electrical energy to be supplied to the ultrasonic transducer in such a manner as to obtain a state in which the biological tissue is incised and/or is degenerated by friction heat attributed to the ultrasonic vibration at the end effector. The processor carries out switching to the second control mode based on satisfaction of a predetermined condition in the first control mode.

The processor acquires a parameter that changes corresponding to a state of the biological tissue and determines whether or not the predetermined condition is satisfied based on the parameter acquired. The processor acquires impedance of a circuit in which the high-frequency current flows as the parameter and the processor determines that the predetermined condition is satisfied based on a state in which the impedance is higher than a predetermined threshold. The processor sets the predetermined threshold in a range from 500 to 1000 S2 inclusive. The processor acquires temperature of the end effector as the parameter and the processor determines that the predetermined condition is satisfied based on a state in which the temperature is higher than a predetermined threshold. The processor acquires impedance of the ultrasonic transducer as the parameter and the processor determines that the predetermined condition is satisfied based on a state in which the impedance is higher than a predetermined threshold. The processor determines that the predetermined condition is satisfied based on a state in which a continuation time of output control in the first control mode is longer than a predetermined threshold. After output control in the second control mode, the processor carries out switching to the first control mode based on satisfaction of a predetermined condition in the second control mode and carries out output control in the first control mode again. The processor repeats the output control in the first control mode and the output control in the second control mode alternately. The first voltage waveform is a waveform of a continuous wave continuously output over time and the second voltage waveform is a burst wave intermittently output. The output is carried out to a parenchymal organ as the biological tissue.

Another aspect of the disclosed technology is directed to a treatment system comprises a power supply apparatus, a return electrode configured to be detachably attached to the power supply and a treatment instrument configured to communicate electrically with the power supply apparatus so as to perform an operation on a biological tissue. The treatment instrument includes a housing, a shaft, and a rod member having an end effector all of which being attached to one another to define the treatment instrument. The end effector transmits a high-frequency current delivered by a first electrical energy to the biological tissue. An ultrasonic transducer is disposed in the treatment instrument. The ultrasonic transducer generates ultrasonic vibration delivered by a second electrical energy to the end effector. The power supply apparatus includes a processor that in a first control mode, the processor supplies the first electrical energy to the end effector with a first voltage waveform about which a crest factor is equal to or lower than 1.5, and controls output of the first electrical energy in such a manner that the biological tissue is degenerated and coagulated by Joule heat generated due to flowing of the high-frequency current in the biological tissue with no discharge occurs when a gap is made between the end effector and the biological tissue. In a second control mode, the processor supplies the first electrical energy to the end effector with a second voltage waveform that generates a discharge between the end effector and the biological tissue and causes the second electrical energy to be supplied to the ultrasonic transducer in such a manner as to obtain a state in which the biological tissue is incised and/or is degenerated by friction heat attributed to the ultrasonic vibration at the end effector. The processor carries out switching to the second control mode based on satisfaction of a predetermined condition in the first control mode.

The return electrode is attached to the power supply via a first cable. The treatment instrument communicates electrically with the power supply apparatus via a second cable. The end effector and the return electrode function as electrodes having potentials different from one another, which causes the high-frequency current to flow between the end effector and the return electrode and transmits the high-frequency current to the biological tissue. The ultrasonic transducer is connected to the rod member inside the housing. The power supply apparatus includes an ultrasonic power supply having a waveform generator, a conversion circuit, a relay circuit, a transformer all of which forms an ultrasonic drive circuit.

While various embodiments of the disclosed technology have been described above, it should be understood that they have been presented by way of example only, and not of limitation. Likewise, the various diagrams may depict an example schematic or other configuration for the disclosed technology, which is done to aid in understanding the features and functionality that can be included in the disclosed technology. The disclosed technology is not restricted to the illustrated example schematic or configurations, but the desired features can be implemented using a variety of alternative illustrations and configurations. Indeed, it will be apparent to one of skill in the art how alternative functional, logical or physical locations and configurations can be implemented to implement the desired features of the technology disclosed herein.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent.

Additionally, the various embodiments set forth herein are described in terms of exemplary schematics, block diagrams, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular configuration.

The invention of the present application is not limited to the embodiment described hereinbefore and can be variously modified in such a range as not to depart from the gist thereof at the stage of implementation. Furthermore, the respective embodiments may be carried out in combination as appropriate as far as possible and combined effects are obtained in this case. Moreover, inventions at various stages are included in the embodiment described hereinbefore and various inventions can be extracted based on appropriate combinations in plural constituent requirements disclosed.

What is claimed is:

1. A treatment system comprising:
a power supply apparatus; and
a treatment instrument configured to communicate electrically with the power supply apparatus so as to perform an operation on a biological tissue wherein the treatment instrument having
an end effector that transmits a high-frequency current delivered by a first electrical energy to the biological tissue, and
an ultrasonic transducer being disposed in the treatment instrument, the ultrasonic transducer generates ultrasonic vibration when a second electrical energy being supplied, the ultrasonic transducer delivers the ultrasonic vibration to the end effector and wherein
the power supply apparatus includes a processor that
in a first control mode, the processor supplies the first electrical energy to the end effector with a first voltage waveform about which a crest factor is equal to or lower than 1.5, and controls output of the first electrical energy in such a manner that the biological tissue is degenerated and coagulated by Joule heat generated due to flowing of the high-frequency current in the biological tissue with no discharge occurs when a gap is made between the end effector and the biological tissue,
in a second control mode, the processor supplies the first electrical energy to the end effector with a second voltage waveform that generates a discharge between the end effector and the biological tissue, and causes the second electrical energy to be supplied to the ultrasonic transducer in such a manner as to obtain a state in which the biological tissue is incised and/or is degenerated by friction heat attributed to the ultrasonic vibration at the end effector, and
the processor carries out switching to the second control mode based on satisfaction of a predetermined condition in the first control mode.

2. The treatment system of claim 1, wherein
the processor acquires a parameter that changes corresponding to a state of the biological tissue and determines whether or not the predetermined condition is satisfied based on the parameter acquired.

3. The treatment system of claim 2, wherein
the processor acquires impedance of a circuit in which the high-frequency current flows as the parameter, and
the processor determines that the predetermined condition is satisfied based on a state in which the impedance is higher than a predetermined threshold.

4. The treatment system of claim 3, wherein
the processor sets the predetermined threshold in a range from 500 to 1000 S2 inclusive.

5. The treatment system of claim 2, wherein
the processor acquires temperature of the end effector as the parameter, and
the processor determines that the predetermined condition is satisfied based on a state in which the temperature is higher than a predetermined threshold.

6. The treatment system of claim 2, wherein
the processor acquires impedance of the ultrasonic transducer as the parameter, and
the processor determines that the predetermined condition is satisfied based on a state in which the impedance is higher than a predetermined threshold.

7. The treatment system of claim 1, wherein
the processor determines that the predetermined condition is satisfied based on a state in which a continuation time of output control in the first control mode is longer than a predetermined threshold.

8. The treatment system of claim 1, wherein
after output control in the second control mode, the processor carries out switching to the first control mode based on satisfaction of a predetermined condition in the second control mode and carries out output control in the first control mode again.

9. The treatment system of claim 8, wherein
the processor repeats the output control in the first control mode and the output control in the second control mode alternately.

10. The treatment system of claim 1, wherein
the first voltage waveform is a waveform of a continuous wave continuously output over time and the second voltage waveform is a burst wave intermittently output.

11. The treatment system of claim 1, wherein
output is carried out to a parenchymal organ as the biological tissue.

12. A treatment system comprising:
a power supply apparatus;
a return electrode configured to be detachably attached to the power supply; and
a treatment instrument configured to communicate electrically with the power supply apparatus so as to perform an operation on a biological tissue, the treatment instrument includes a housing, a shaft, and a rod member having an end effector all of which being attached to one another to define the treatment instrument and wherein
the end effector transmits a high-frequency current delivered by a first electrical energy to the biological tissue, and
an ultrasonic transducer being disposed in the treatment instrument, the ultrasonic transducer generates ultrasonic vibration delivered by a second electrical energy to the end effector and wherein
the power supply apparatus includes a processor that
in a first control mode, the processor supplies the first electrical energy to the end effector with a first voltage waveform about which a crest factor is equal to or lower than 1.5, and controls output of the first electrical energy in such a manner that the biological tissue is degenerated and coagulated by Joule heat generated due to flowing of the high-frequency current in the biological tissue with no discharge occurs when a gap is made between the end effector and the biological tissue,
in a second control mode, the processor supplies the first electrical energy to the end effector with a second voltage waveform that generates a discharge between the end effector and the biological tissue, and causes the second electrical energy to be supplied to the ultrasonic transducer in such a manner as to obtain a state in which the biological tissue is incised and/or is degenerated by friction heat attributed to the ultrasonic vibration at the end effector, and
the processor carries out switching to the second control mode based on satisfaction of a predetermined condition in the first control mode.

13. The treatment system of claim 12, wherein the return electrode is attached to the power supply via a first cable.

14. The treatment system of claim 12, wherein the treatment instrument communicates electrically with the power supply apparatus via a second cable.

15. The treatment system of claim 12, wherein the end effector and the return electrode function as electrodes having potentials different from one another, which causes the high-frequency current to flow between the end effector and the return electrode and transmits the high-frequency current to the biological tissue.

16. The treatment system of claim 12, wherein the ultrasonic transducer is connected to the rod member inside the housing.

17. The treatment system of claim 12, wherein the power supply apparatus includes an ultrasonic power supply having a waveform generator, a conversion circuit, a relay circuit, a transformer all of which forms an ultrasonic drive circuit.
